(19) United States
(12) Patent Application Publication
(10) Pub. No.: US 2018/0158261 A1
(43) Pub. Date: Jun. 7, 2018

(54) SYSTEM AND METHOD FOR SENSING LUBRICANT AND ENGINE HEALTH

(71) Applicant: General Electric Company, Schenectady, NY (US)

(72) Inventors: Pradheepram Ottikkutti, Lawrence Park, PA (US); Najeeb M. Kuzhiyil, McKinney, TX (US); Radislav Alexandrovich Potyrailo, Niskayuna, NY (US)

(21) Appl. No.: 15/884,889

(22) Filed: Jan. 31, 2018

Related U.S. Application Data
(63) Continuation-in-part of application No. 14/585,690, filed on Dec. 30, 2014.
Application No. 14/585,690 is a continuation-in-part of application No. 11/560,476, filed on Nov. 16, 2006, now Pat. No. 9,589,686.
Application No. 11/560,476 is a continuation-in-part of application No. 12/325,653, filed on Dec. 1, 2008, now abandoned.
Application No. 12/325,653 is a continuation-in-part of application No. 12/824,436, filed on Jun. 28, 2010.
Application No. 12/824,436 is a continuation-in-part of application No. 12/827,623, filed on Jun. 30, 2010, now Pat. No. 8,936,191.
Application No. 12/827,623 is a continuation-in-part of application No. 12/977,568, filed on Dec. 23, 2010, now abandoned.
Application No. 12/977,568 is a continuation-in-part of application No. 13/331,003, filed on Dec. 20, 2011, now Pat. No. 9,045,973.
Application No. 13/331,003 is a continuation-in-part of application No. 13/484,674, filed on May 31, 2012, now Pat. No. 9,052,263.
Application No. 13/484,674 is a continuation-in-part of application No. 12/424,016, filed on Apr. 15, 2009, now Pat. No. 8,364,419.
Application No. 14/585,690 is a continuation-in-part of application No. 13/538,570, filed on Jun. 29, 2012, now Pat. No. 9,538,657.
Application No. 13/538,570 is a continuation-in-part of application No. 13/558,499, filed on Jul. 26, 2012, now Pat. No. 9,195,925.
Application No. 13/558,499 is a continuation-in-part of application No. 13/630,939, filed on Sep. 28, 2012, now Pat. No. 9,389,260.
Application No. 13/630,939 is a continuation-in-part of application No. 13/630,954, filed on Sep. 28, 2012, now Pat. No. 9,147,144.
Application No. 13/630,954 is a continuation-in-part of application No. 13/630,587, filed on Sep. 28, 2012, now Pat. No. 9,658,178.
Application No. 13/630,587 is a continuation-in-part of application No. 13/630,739, filed on Sep. 28, 2012, now Pat. No. 9,176,083.
Application No. 13/630,739 is a continuation-in-part of application No. 13/729,800, filed on Dec. 28, 2012, now Pat. No. 9,097,639.
Application No. 13/729,800 is a continuation-in-part of application No. 13/729,851, filed on Dec. 28, 2012, now Pat. No. 9,261,474.
Application No. 13/729,851 is a continuation-in-part of application No. 13/838,884, filed on Mar. 15, 2013, now Pat. No. 9,389,296.
Application No. 13/838,884 is a continuation-in-part of application No. 14/031,951, filed on Sep. 19, 2013, now Pat. No. 9,037,418.
Application No. 14/031,951 is a continuation-in-part of application No. 14/031,965, filed on Sep. 19, 2013, now Pat. No. 8,990,025.
Application No. 14/031,965 is a continuation-in-part of application No. 14/532,168, filed on Nov. 4, 2014, now Pat. No. 9,536,122.
(60) Provisional application No. 62/459,806, filed on Feb. 16, 2017.
(60) Provisional application No. 61/987,853, filed on May 2, 2014.

(51) Int. Cl. G07C 5/08; G07C 5/00; F16N 29/04; G01N 33/28

(52) U.S. Cl. CPC G07C 5/0816; G07C 5/006; B60W 2400/00; F16N 29/04; G01N 33/2888; G07C 5/0841

(57) ABSTRACT

A system includes a sensor configured to be in contact with lubricant within an engine of a vehicle system. The sensor includes a sensing region circuit that is configured to generate stimuli at different times during an operational life of the engine. The system also includes one or more processors configured to receive signals from the sensor. The signals are representative of responses of the lubricant to the stimuli. The one or more processors are configured to analyze the responses and determine a characteristic of the lubricant that represents one or more of a total base number (TBN) or a total acid number (TAN) of the lubricant. The one or more processors are configured to determine an unhealthy state of one or more of the engine or the lubricant based on the characteristic of the lubricant that is determined.

### CROSS-REFERENCE TO RELATED APPLICATIONS

This application claims priority to U.S. Provisional Application No. 62/459,806, which was filed on 16 Feb. 2017. This application also is a continuation-in-part of U.S. patent application Ser. No. 14/585,690, which was filed on 30 Dec. 2014 (the “'690 application”).

The '690 application claims priority to U.S. Provisional Patent Application No. 61/987,853 filed on 2 May 2014, and is a continuation-in-part of the following applications: U.S. patent application Ser. No. 11/560,476, filed on 16 Nov. 2006 (now U.S. Pat. No. 9,589,686, issued on 7 Mar. 2017); U.S. patent application Ser. No. 12/325,653, filed on 1 Dec. 2008; U.S. patent application Ser. No. 12/824,436, filed on 28 Jun. 2010; U.S. patent application Ser. No. 12/827,623, filed on 30 Jun. 2010 (now U.S. Pat. No. 8,936,191, issued on 20 Jan. 2015); U.S. patent application Ser. No. 12/977,568, filed on 23 Dec. 2010; U.S. patent application Ser. No. 13/331,003, filed on 20 Dec. 2011 (now U.S. Pat. No. 9,045,973, issued 2 Jun. 2015); U.S. patent application Ser. No. 13/484,674, filed on 31 May 2012 (now U.S. Pat. No. 9,052,263, which issued on 9 Jun. 2015, which is a continuation-in-part of U.S. patent application Ser. No. 12/424,016, filed on 15 Apr. 2009, now U.S. Pat. No. 8,364,419, issued on 29 Jan. 2013); U.S. patent application Ser. No. 13/538,570, filed on 29 Jun. 2012 (now U.S. Pat. No. 9,538,657, issued on 3 Jan. 2017); U.S. patent application Ser. No. 13/558,499, filed on 26 Jul. 2012 (now U.S. Pat. No. 9,195,925, issued on 24 Nov. 2015); U.S. patent application Ser. No. 13/630,939 (now U.S. Pat. No. 9,389,260, issued on 12 Jul. 2016), Ser. No. 13/630,954 (now U.S. Pat. No. 9,147,144, issued on 29 Sep. 2015), Ser. No. 13/630,587 (now U.S. Pat. No. 9,658,178, issued on 23 May 2017), and Ser. No. 13/630,739 (now U.S. Pat. No. 9,176,083, issued on 3 Nov. 2015), all filed on 28 Sep. 2012; U.S. patent application Ser. No. 13/729,800 (now U.S. Pat. No. 9,097,639, issued on 4 Aug. 2015) and Ser. No. 13/729,851 (now U.S. Pat. No. 9,261,474, issued on 16 Feb. 2016), both filed on 28 Dec. 2012; U.S. patent application Ser. No. 13/838,884, filed on 15 Mar. 2013 (now U.S. Pat. No. 9,389,296, issued on 12 Jul. 2016); U.S. patent application Ser. No. 14/031,951 (now U.S. Pat. No. 9,037,418, issued on 19 May 2015) and Ser. No. 14/031,965 (now U.S. Pat. No. 8,990,025, issued on 24 Mar. 2015), both filed on 19 Sep. 2013; and U.S. patent application Ser. No. 14/532,168, filed on 4 Nov. 2014 (now U.S. Pat. No. 9,536,122, issued on 3 Jan. 2017).

The entirety of each of the aforementioned patent applications and patents is incorporated herein by reference.

### FIELD

One or more embodiments are disclosed that relate to systems and methods for sensing lubricant health and/or lubricant consumption in engines.

### BACKGROUND

Many industrial machines include equipment or assemblies (e.g., engines) that use lubricants (e.g., oil) to operate. It is desirable to monitor a condition of the lubricant to ensure that the lubricant is replaced or replenished before severe and permanent damage is sustained by the machine. In addition to lubricants, machines may use other industrial fluid such as fuels, hydraulic media, drive fluids, power steering fluids, power brake fluids, drilling fluids, oils, insulating fluids, heat transfer fluids, or the like. Such fluids allow efficient and safe operation of machinery in transportation, industrial, locomotive, marine, automotive, construction, medical, and other applications.

The quality of a lubricant may deteriorate over time due to the introduction of contaminants and/or aging of the lubricant. Generally, lubricants contain additives that provide increased resilience. Such additives also break down in service over time. As the additives deplete, acidic components such as by-products from the degradation of the additive and/or the lubricant due to aging, may be introduced into the lubricant. In case of engine oil, combustion products that ingress into the oil sump introduce acidic components into the oil. The acidic components can reduce the effectiveness and performance of the lubricant. In order to neutralize acidic components, engine oils are formulated with basic (alkaline) additives. The quantity of alkaline additives remaining in the engine oil (Total Base Number or TBN) is a measure of its health.

Current technique employed for determining the quality of engine oils is to collect oil samples every fifteen to thirty days and to analyze in a chemical laboratory. The process of collection of oil samples, their storage and transport to a chemical laboratory for analysis may add relatively large variability in measured results in initially identical oil samples. The typical lead time for results is another fifteen to thirty days. This process is inefficient to provide timely information on the condition of the oil and hence on the health of the engine and also to provide real time information on oil consumption rate. Use of this fast response, real-time oil quality/health monitoring eliminates possible mistakes or mishaps caused by mishandled or mislabeled oil samples, oil sample bottles lost in transit, analysis reports that are incorrectly numbered or mislabeled, the normal 15 to 30 days required to complete the oil analysis (being too long), and resulting in engine failure and road failure of the vehicle if the quality of the oil (used) is very close to an end-of-life limit, etc.

### BRIEF DESCRIPTION

In one embodiment, a system includes a sensor configured to be in contact with oil within an engine of a vehicle system. The sensor includes a sensing region circuit that is configured to generate stimuli at different times during an operational life of the engine. The system also includes one or more processors configured to receive signals from the sensor. The signals are representative of responses of the oil to the stimuli. The one or more processors are configured to analyze the responses and determine a characteristic of the oil that represents one or more of a total base number (TBN) or a total acid number (TAN) of the oil. The one or more processors are configured to determine an unhealthy state of one or more of the engine or the oil based on the characteristic of the oil that is determined.

In one embodiment, a method includes placing a sensor in contact with oil within an engine of a vehicle system, generating stimuli at a sensing region circuit of the sensor during an operational life of the engine, receiving signals from the sensor, the signals representative of responses of the oil to the stimuli, analyzing the responses to determine a characteristic of the oil that represents one or more of a total base number (TBN) or a total acid number (TAN) of the oil, and determining an unhealthy state of one or more of the engine or the oil based on the characteristic TBN and/or TAN of the oil that is determined.

In one embodiment, a system includes a sensor configured to be in contact with lubricating oil within a rotating equipment of a system. The sensor is configured to generate detectable stimuli at different times during an operational life of the rotating equipment. The system also includes one or more processors configured to receive signals from the sensor. The signals are representative of responses of the oil to the stimuli. The one or more processors are configured to analyze the responses and determine a characteristic of the oil that represents one or more of a total base number (TBN) or a total acid number (TAN) of the oil. The one or more processors are configured to determine an unhealthy state of one or more of the engine or the oil based on the characteristic of the oil that is determined.

In one embodiment, a method includes placing a sensor in contact with oil within an engine of a vehicle system, generating stimuli at a sensing region circuit of the sensor during an operational life of the engine, receiving signals from the sensor, the signals representative of responses of the oil to the stimuli, analyzing the responses to determine a characteristic of the oil that represents one or more of a total base number (TBN) or a total acid number (TAN) of the oil, and determining health state of one or more of the engine or the oil based on the characteristic of the oil that is determined.

### BRIEF DESCRIPTION OF THE DRAWINGS

FIG. 1 illustrates one embodiment of a sensing system.

FIG. 2 illustrates a non-limiting example of a design of the resonant sensor shown in FIG. 1.

FIG. 3 illustrates a flowchart of one embodiment of a method for monitoring lubricant (e.g., oil) health.

FIG. 4 illustrates a flowchart of one embodiment of a method for monitoring lubricant (e.g., oil) health.

FIG. 5 illustrates measurements of TBN according to one example.

FIG. 6 illustrates measurements of TAN according to one example.

FIG. 7 illustrates a first derivative of TBN with respect to time according to one example.

FIG. 8 illustrates a first derivative of TAN with respect to time according to one example.

FIG. 9 illustrates a second derivative of TBN with respect to time according to one example.

FIG. 10 illustrates a second derivative of TAN with respect to time according to one example.

FIG. 11 illustrates examples of normal and acceptable limits of TBN measurements.

FIG. 12 illustrates examples of normal and acceptable limits of TAN measurements.

FIG. 13 illustrates examples of normal and acceptable limits of the first derivative of TBN.

FIG. 14 illustrates examples of normal and acceptable limits of the second derivative of TBN.

FIG. 15 illustrates examples of normal and acceptable limits of the first derivative of TAN.

FIG. 16 illustrates examples of normal and acceptable limits of the second derivative of TAN.

### DETAILED DESCRIPTION

Embodiments described herein include various systems, assemblies, devices, apparatuses, and methods that may be used in a connection with obtaining one or more measurements of a machine. The measurement(s) may be representative or indicative of an operative condition of the machine. The operative condition of the machine may refer to an operative condition of the machine as a whole or an operative condition of a component (e.g., element, assembly, or sub-system) of the machine. The operative condition can relate to a present state or ability of the component and/or a future state or ability. For example, the measurement may indicate that a component is not functioning in a sufficient manner, is damaged, is likely to be damaged if it continues to operate in a designated manner, is not likely to perform appropriately under designated circumstances, and/or is likely to cause damage to other components of the machine.

Internal combustion engine oils can be made with additives to neutralize acidic combustion products that get into the oil. In modern internal combustion engines with medium to high rates of exhaust gas recirculation (EGR), more acidic combustion products are likely to enter engine oil. These products can rapidly deplete the total base number (TBN) of the oil. One or more embodiments of the inventive subject matter described herein repeatedly measure (such as by continuously measuring) the total base number of engine oil using a sensor to detect and measure abnormal rate of oil consumption in the engine. These measurements can be used for engine prognostics and engine protection.

One or more embodiments of the oil sensor described herein measures the TBN of the oil in a real-time basis and sends data representative of the measurements to a prognosis device. The prognosis device compares a rate of TBN depletion with a previously determined or calibrated depletion rate. The predetermined rate may be selected from several different predetermined rates, with the different predetermined rates being associated with different engine operating conditions (e.g., idle, low/medium/full power, operating within a tunnel, operating at different altitudes, etc.), different ages of the engine, and/or different ambient conditions. The predetermined rate that is used to compare to the measured depletion rate is selected from the different predetermined rate by matching the operating conditions in which the engine is or has been operating with the operating conditions associated with one or more of the predetermined rates.

When the rate of TBN depletion is higher (e.g., faster) than normal or outside of a set range (as determined from or based on the predetermined rate), the engine may be identified as needing inspection for excessive engine wear, fuel system wear, specification and quality of lube oil being used, and/or lubricating oil system functionality. For example, decreases or depletion of TBN may indicate that the engine needs inspection, repair, and/or maintenance. TBN is depleted from lubricating oil when the oil comes into contact with acidic products of the combustion process of the engine (e.g., which can arise from high wear of the engine cylinders, liners, and/or piston rings). Changes in the TBN value (e.g., below a designated threshold, decreasing at rates that are faster than a designated rate, and/or having second derivatives that change faster than designated second derivatives) can indicate a need for repair, inspection, and/or maintenance of the lubricant and/or engine. Repair, inspection, and/or maintenance of the engine may be automatically implemented responsive to determining that the TBN depletion rate is faster than desired.

Similarly, an increase in total acid number (TAN) of the oil may be measured (e.g., in real time) using the sensor, and data representative of the measurements can be used to determine engine wear, fuel system wear, and/or lube oil system prognostics. TAN is a measurement of an amount of acid content in oil. A similar approach to comparing normal and abnormal rates of increase of TAN in the oil and to flag engines for inspection may be used. For example, increases in TAN (e.g., above a designated threshold, at rates that are faster than a designated rate, and/or having second derivatives that are faster than designated second derivatives) may indicate that the engine needs inspection, repair, and/or maintenance. TAN increases in the oil when the oil comes into contact with acidic products of the combustion process of the engine (e.g., which can arise from high wear of the engine cylinders, liners, and/or piston rings).

In addition or instead of the rate of depletion of TBN or an increase of TAN, a first derivative or a higher derivative of the rate of depletion of TBN or an increase of TAN can be used. The TBN and/or TAN of engine lubricating oil can be continuously monitored and measured values recorded periodically and frequently (e.g., every hour) as dictated by the specific application and/or duty cycle of the engine. Using a self-learning or a machine-learning algorithm, behavior of the oil for each engine will be mapped. For each engine, individual TBN and/or TAN readings, the rates of change, and/or the rate of change of rate of change (e.g., the first and second derivatives of the rate of change) of TBN and/or TAN can be determined. The systems and methods determine whether the values are within the normal or healthy values by comparing the measured values with established normal values and ranges. Responsive to the amounts of TBN or TAN, the rates of TBN or TAN depletion, and/or the second derivatives of depletion rates are outside the healthy-engine range, one or more responsive actions can be automatically implemented to check the engine and the relevant engine components. At least one technical effect of the subject matter described herein includes the ability to replace or replenish lubricant in a machine due to the measured TBN and/or TAN prior to damage to the machine occurring.

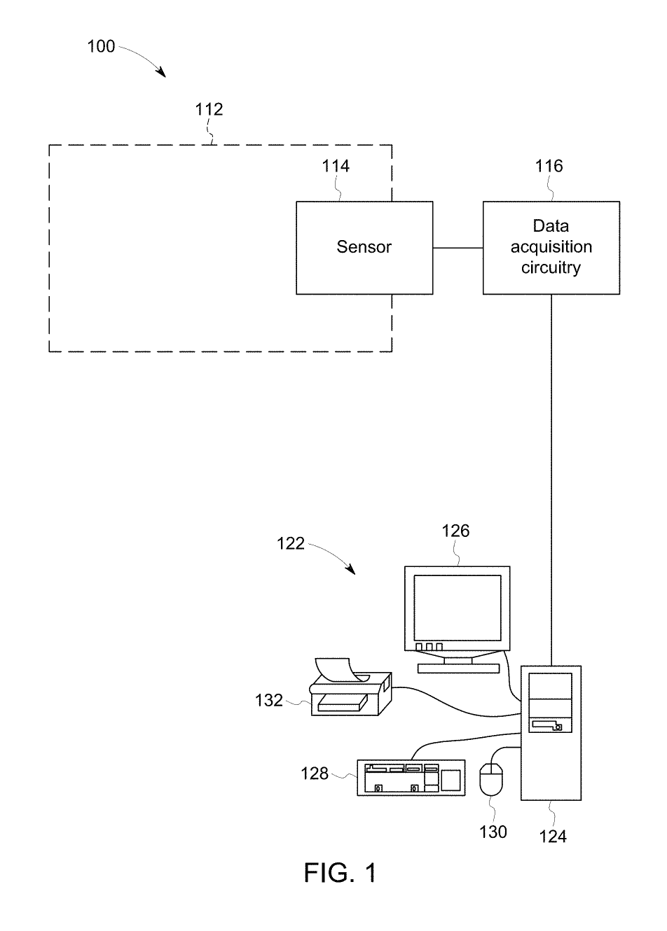

FIG. 1 illustrates one embodiment of a sensing system 100. The sensing system 100 examines a fluid in contact with the sensing system 100. This fluid may be engine oil. The system 100 may include a fluid reservoir 112 for holding the fluid and one or more sensors 114 at least partially disposed in, on, or within the fluid reservoir 112. Alternatively, the sensor 114 may be set in a flow path of the fluid outside of the reservoir 112, such as coupled to in-line connectors in fluid communication with the fluid reservoir that define a flow path. In one embodiment, the sensor 114 may provide continuous monitoring of the fluid within the reservoir or flow path.

Suitable fluids may include hydrocarbon fuels and lubricants. Suitable lubricants may include engine oil, gear oil, hydraulic fluid, lubricating oils, synthetic based lubricants, lubricating fluids, greases, silicones, and the like. Suitable fuels may include gasoline, diesel fuel, jet fuel or kerosene, bio-fuels, petrodiesel-biodiesel fuel blends, natural gas (liquid or compressed), and fuel oils. Still other fluids may be insulating oils in transformers, solvents, or mixtures of solvents. Still other fluids may be included with correspondingly appropriate sensor parameters, such as water, air, engine exhaust, biologic fluids, and organic and/or vegetable oils. The fluid may be a liquid, or may be in a gaseous phase. Further contemplated are multiphase compositions. The fluids may be disposed in and/or used in an operating machine, such as a movable vehicle or a wind turbine.

Nonlimiting examples of design of the sensor 114 include various sensor designs. Depending on the measured parameter of oil and the concentration level of the measured parameter, an oil sensor can be a capacitor sensor, a resistor sensor, a non-resonant impedance sensor, a resonant impedance sensor, an electro-mechanical resonator sensor (e.g., tuning fork, cantilever sensor, acoustic device sensor), a thermal sensor, an optical sensor, an acoustic sensor, a photoacoustic sensor, a near-infrared sensor, a ultraviolet sensor, an infrared sensor, a visible light sensor, fiber-optic sensor, and reflection sensor, a multivariable sensor, or a single-output sensor. The sensor may generate electrical or optical stimuli to the measured oil.

In one embodiment, the sensor 114 may detect characteristics or properties of the fluid via a resonant impedance spectral response. One or more of the inductor-capacitor-resistor resonant circuits (LCR resonators) may measure the resonant impedance spectral response. As opposed to simple impedance measurements, the disclosed embodiments probe the sample with at least one resonant electrical circuit. The resonant impedance spectrum of the sensor 114 in proximity to the fluid varies based on sample composition and/or components and/or temperature. The measured resonant impedance values Z′ (which may be the real part of resonant impedance, Zre) and Z″ (which may be the imaginary part of resonant impedance, Zim) reflect the response of the fluid (for example, the portion of the fluid in proximity to the sensor) to a stimulus of the electric field of the resonant electrical circuit.

The electrical field may be applied by the sensor 114 via electrodes. The electrodes may be in direct or indirect electrical contact with the sample. For example, a sensor 114 may be a combination of a sensing region and associated circuits. The sensing region may be either bare or coated with a protective dielectric layer or a sensing layer. In each of the disclosed cases, the sensing region may be in operational contact with a fluid. In such embodiments, the sensor circuits may not contact the fluid directly. An example of indirect electrical contact with the sample may be when a sensing electrode structure is coated with a dielectric protective coating and when the electric field that may be generated between the electrodes interacts with the fluid after penetrating through the dielectric protective coating. A suitable dielectric protective coating may be conformally applied to the electrode.

Suitable sensors may include single use or multi-use sensors. A suitable multi-use resonant sensor may be a re-usable sensor that may be used during the lifetime of a system in which it may be incorporated into. In one embodiment, the resonant sensor may be a single use sensor that may be used during all or part of a reaction or process. For example, the resonant sensor may include one or more pairs of electrodes and one or more tuning elements, e.g., a resistor, a capacitor, an inductor, a resonator, impedance transformer, or combinations of two or more thereof to form an inductor-capacitor-resistor (LCR) resonant circuit operated at one or more resonant frequencies. In certain embodiments, different resonant circuits of a plurality of resonant circuits of a resonant sensor may be configured to resonate at different frequencies. Different frequencies may be selected to be across the dispersion profile of the measured fluid composition. The dispersion profile may depend on the dielectric properties of the fluid composition on the probing frequency. Various components of the fluid have different dispersion profiles. When measured at multiple resonance frequencies, concentrations of different components of the fluid may be determined.

Data from the resonant sensor 114 may be acquired via data acquisition circuitry 116, which may be associated with the sensor or which may be associated with a control system, such as a controller or workstation 122 including data processing circuitry, where additional processing and analysis may be performed. The controller or workstation may include one or more wireless or wired components, and may also communicate with the other components of the system. Suitable communication models include wireless or wired. At least one suitable wireless model includes radio frequency devices, such as radio frequency identification (RFID) wireless communications. Other wireless communication modalities may be used based on application specific parameters. For example, where there may be electromagnetic field (EMF) interference, certain modalities may work where others may not. The data acquisition circuitry optionally can be disposed within the sensor 114. Other suitable locations may include disposition being within the workstation. Further, the workstation can be replaced with a control system of the whole process where the resonant sensor and its data acquisition circuitry may be connected to the control system of process.

The data acquisition circuitry may be in the form of a sensor reader, which may be configured to communicate wirelessly or wired with the fluid reservoir and/or the workstation. For example, the sensor reader may be a battery-operated device and/or may be powered using energy available from the main control system or by using harvesting of energy from ambient sources (light, vibration, heat, or electromagnetic energy).

Additionally, the data acquisition circuitry may receive data from one or more resonant sensors 114 (e.g., multiple sensors formed in an array or multiple sensors positioned at different locations in or around the fluid reservoir). The data may be stored in short or long-term memory storage devices, such as archiving communication systems, which may be located within or remote from the system and/or reconstructed and displayed for an operator, such as at the operator workstation. The sensors may be positioned on or in fuel or fluid reservoirs, associated piping components, connectors, flow-through components, and any other relevant process components. The data acquisition circuitry may include one or more processors for analyzing the data received from the sensor 114. For example, the one or more processors may be one or more computer processors, controllers (e.g., microcontrollers), or other logic-based devices that perform operations based on one or more sets of instructions (e.g., software). The instructions on which the one or more processors operate may be stored on a tangible and non-transitory computer readable storage medium, such as a memory device. The memory device may include a hard drive, a flash drive, RAM, ROM, EEPROM, and/or the like. Alternatively, one or more of the sets of instructions that direct operations of the one or more processors may be hard-wired into the logic of the one or more processors, such as by being hard-wired logic formed in the hardware of the one or more processors.

In addition to displaying the data, the operator workstation may control the above-described operations and functions of the system. The operator workstation may include one or more processor-based components, such as general purpose or application-specific computers 124. In addition to the processor-based components, the computer may include various memory and/or storage components including magnetic and optical mass storage devices, internal memory, such as RAM chips. The memory and/or storage components may be used for storing programs and routines for performing the techniques described herein that may be executed by the operator workstation or by associated components of the system. Alternatively, the programs and routines may be stored on a computer accessible storage and/or memory remote from the operator workstation but accessible by network and/or communication interfaces present on the computer. The computer may also comprise various input/output (I/O) interfaces, as well as various network or communication interfaces. The various I/O interfaces may allow communication with user interface devices, such as a display 126, keyboard 128, electronic mouse 130, and printer 132, that may be used for viewing and inputting configuration information and/or for operating the imaging system. Other devices, not shown, may be useful for interfacing, such as touchpads, heads up displays, microphones, and the like. The various network and communication interfaces may allow connection to both local and wide area intranets and storage networks as well as the Internet. The various I/O and communication interfaces may utilize wires, lines, or suitable wireless interfaces, as appropriate or desired.

The sensor 114 may include a plurality of resonant circuits that may be configured to probe the fluid in the fluid reservoir with a plurality of frequencies. The fluid reservoir may be a reservoir bound by the engineered fluid-impermeable walls or by naturally formed fluid-impermeable walls or by the distance of the electromagnetic energy emitted from the sensor region to probe the fluid. Further, the different frequencies may be used to probe a fluid sample at different depths. In certain embodiments, an integrated circuit memory chip may be galvanically coupled to the resonant sensor. The integrated circuit memory chip may contain different types of information. Non-limiting examples of such information in the memory of the integrated circuit chip include calibration coefficients for the sensor, sensor lot number, production date, and/or end-user information. In another embodiment, the resonant sensor may comprise an interdigital structure that has a fluid-sensing region.

In certain embodiments, when an integrated circuit memory chip may be galvanically coupled to the resonant sensor, readings of the sensor response may be performed with a sensor reader that contains circuitry operable to read the analog portion of the sensor. The analog portion of the sensor may include resonant impedance. The digital portion of the sensor may include information from the integrated circuit memory chip.

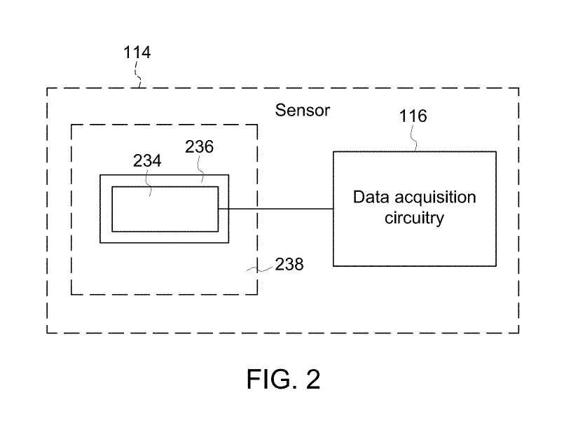

FIG. 2 illustrates a non-limiting example of a design of the resonant sensor 114. A sensing electrode structure 234 of the sensor may be connected to the tuning circuits and the data acquisition circuitry 116. The sensing electrode structure 234 can be bare and in direct contact with the fluid. Alternatively, the sensing electrode structure can be coated with a protective or sensing coating 236. The sensing electrode structure, without or with the protective or sensing coating, forms a sensing region 238. The coating may be applied conformably, and may be a dielectric material. The sensing electrode structure, without or with the protective coating that forms the sensing region, may operationally contact a fluid. The fluid contains the analyte or contaminant(s). The sensing electrode structure may be either without (bare) or with a protective coating.

A bare sensing electrode structure may generate an electric field between the electrodes that interacts directly with the fluid. A dielectric protective coated sensing electrode structure may generate an electric field that is between the electrodes that interacts with the fluid after penetrating through the dielectric protective coating. In one embodiment, the coating may be applied onto electrodes to form a conformal protective layer having the same thickness over all electrode surfaces and between electrodes on the substrate. Where a coating has been applied onto electrodes to form a protective layer, it may have a generally constant or variable final thickness over the substrate and sensor electrodes on the substrate. In another embodiment, a substrate simultaneously serves as a protective layer when the electrodes are separated from the fluid by the substrate. In this scenario, a substrate has electrodes on one side that do not directly contact the fluid, and the other side of the substrate does not have electrodes that face the fluid. Detection of the fluid may be performed when the electric field from the electrodes penetrates the substrate and into the fluid. Suitable examples of such substrate materials may include ceramic, aluminum oxide, zirconium oxide, and others. The coating changes as the concentration of contaminants in the lubricant bind to, coupled with, diffuse into, etc., the coating. As the concentration of contaminants in or on the coating changes, the frequencies at which the circuit in the sensor resonates changes. The change in resonance of the circuit can be used to identify and/or measure the level or amount of contaminants in the lubricant. Alternatively, no coating may be used, and the frequencies at which the resonant circuit of the sensor resonates may change based on the concentration of contaminants in the electric field between the electrodes. Alternatively, no coating may be used, and Fp, Zp and other responses from the sensor may change based on the concentration of contaminants in the electric field between the electrodes.

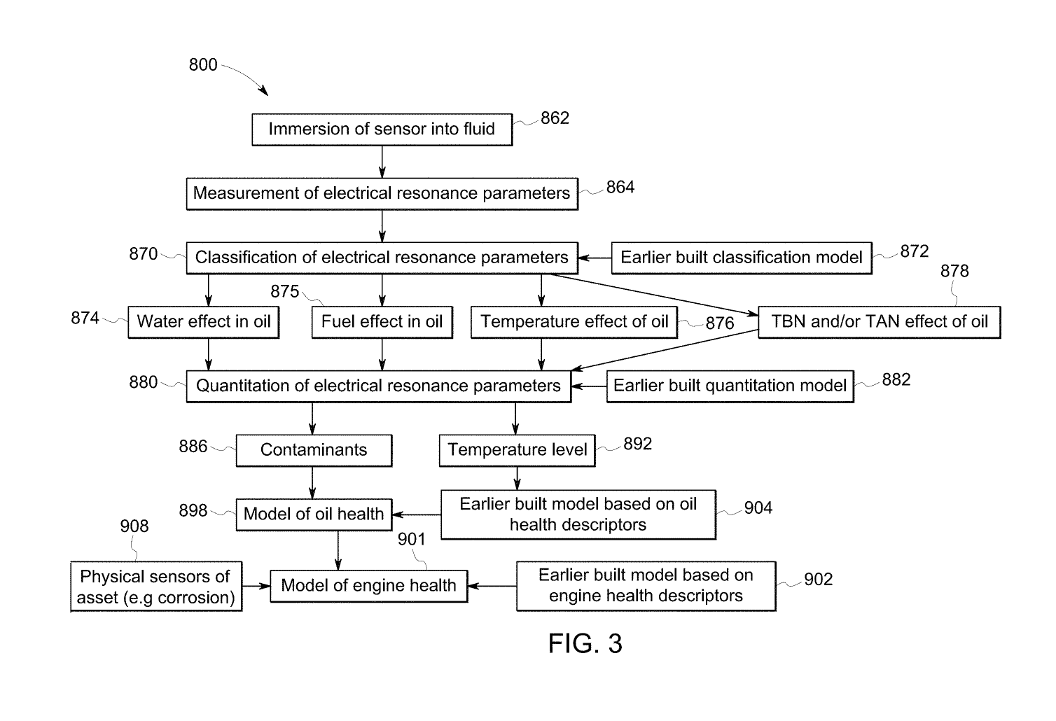

FIG. 3 illustrates a flowchart of one embodiment of a method 800 for monitoring lubricant (e.g., oil) health. At 862, the sensor is at least partially immersed into an oil. At 864, measurement of electrical resonance parameters of the resonance spectra at several resonances of the sensor is performed. For quantitation of contamination of engine oil by water, fuel leaks, and levels of TBN and/or TAN with a sensor, the sensor may be placed into operational contact with the fluid at 862. In a specific embodiment, the resonant impedance spectra Ž(ƒ)=Zᵣₑ(ƒ)+jZᵢₘ(ƒ) of a sensor may be determined at 864. For example, the parameters from the measured Ž(ƒ) spectra such as the frequency position Fₚ and magnitude Zₚ of Zᵣₑ(f) and the resonant F₁ and antiresonant F₂ frequencies, their magnitudes Z₁ and Z₂ of Zᵢₘ(f), and zero-reactance frequency F_{Z} of Zᵢₘ(f), may be calculated. In another embodiment, the electrical resonance parameters may include capacitance parameters of the sensor in operational contact with the fluid, instead or in addition to impedance parameters.

At 870, the electrical resonance parameters are classified. This may be done using a determined classification model at 872 to assess, for example, one or more of water effects (e.g., at 874), fuel effects (e.g., at 875), temperature effects (e.g., at 876), and/or TBN and/or TAN effects (e.g., at 878). At 880, quantitation of the electrical resonance parameters may be performed using a predetermined, earlier saved quantitation model (e.g., at 882). At 886, a determination of components in the oil, such as water, fuel, soot, wear metal particles (e.g., at 890), as well as the temperature (e.g., at 892), prediction of the oil health (e.g., at 898) and the engine health (e.g., at 901). Optionally, the amount of TBN and/or TAN may be determined at 886. This may be done by using one or more of determined engine health descriptors (e.g., at 902) and oil health descriptors (e.g., at 904), as well as inputs from any additional sensors (e.g., at 908). Suitable additional sensors may include those sensing corrosion, temperature, pressure, system (engine) load, system location (e.g., by GPS signal), equipment age calculator, pH, and the like.

For example, in one embodiment, a sensor system may be an electrical resonator that may be excited with a wired or wireless excitation and where a resonance spectrum may be collected and analyzed to extract at least four parameters that may be further processed upon auto scaling or mean centering of the parameters and to quantitatively determine properties of the oil. The properties of the oil that are determined via analyzing the resonance impedance spectrum may include the concentration of water, acid, and/or fuel in engine oil, and the properties may be used to predict the remaining life of the engine oil and/or the remaining life of the engine in which the oil is disposed. The spectral parameters of the resonance spectrum such as Fₚ, Zₚ, F_{z}, F₁, F₂, Z₁, and Z₂ or the whole resonance spectrum with a single or multiple resonators can be used for data processing.

The classification model 872 may be built using the predicted contributions of the spectral parameters for an uncontaminated fluid and for fluid contamination using previously determined component effects and their corresponding spectral parameters. Based on previously or empirically determined effects of components on a particular fluid, the resonance parameters, both real and imaginary, may be changed and/or affected in a quantifiable manner if specific components of interest are present. Further, based on the measured parameters, a concentration of a particular component may also be predicted, and multi-component models may be generated. The disclosed techniques may be used to sense a suitable fluid and to build a component and environmental effect model.

Measurements of the resonant impedance of sensors may be performed with a network analyzer (Agilent) or a precision impedance analyzer (Agilent), under computer control using LabVIEW. Collected resonant impedance data may be analyzed using KaleidaGraph (Synergy Software, Reading, Pa.) and PLS Toolbox (Eigenvector Research, Inc., Manson, Wash.) operated with Matlab (The Mathworks Inc., Natick, Mass.). In another embodiment, measurements of the resonant impedance of sensors may be performed with a mini network analyzer or an integrated circuit impedance analyzer.

By using multivariate analysis of calculated parameters of Ž(ƒ) spectra, classification of analyte may be performed. Suitable analysis techniques for multivariate analysis of spectral data from the multivariable sensors may include Principal Components Analysis (PCA), Independent Component Analysis (ICA), Linear Discriminant Analysis (LDA), and Flexible Discriminant Analysis (FDA). PCA may be used to discriminate between different vapors using the peptide-based sensing material.

By using multivariate analysis, also calculations of non-resonant impedance spectra or optical spectra may be performed.

The one or more processors are configured to analyze the resonant spectral response and determine properties of the fluid. For example, in one embodiment, the one or more processors may be configured to determine a concentration of potassium hydroxide or other basic components in order to determine the TBN of the oil. As another example, the one or more processors may be configured to determine a concentration of acids in order to determine the TAN of the oil.

In one embodiment, measurements of the sensor installed in an asset are correlated to TAN/TBN in the following manner. First, the sensor is calibrated to different levels of TAN and/or TBN. For such calibration, the sensor is exposed to samples related to known levels of TAN and/or TBN. Such sensor exposure allows the sensor to produce responses that are different to different known levels of TAN and/or TBN. As described above, a known quantity of potassium hydroxide can be placed into oil that is examined by the sensor to measure the TBN of the oil. A known quantity of an acid that is placed into oil (e.g., one or more naphthenic acids, such as carboxylic acid) can be examined by the sensor to measure the TAN of the oil.

The response of the sensor may have peaks at frequencies associated with the presence of TAN in the sample and/or associated with the presence of TBN in the sample. The size or magnitude of the peaks, and/or the frequencies at which the peaks occur, can indicate different concentrations of TAN and/or TBN in the sample. The calibration is complete when a correlation between the different known levels of TAN and/or TBN and the corresponding responses from the sensor is established. Such correlation is known as a calibration function. This calibration function unambiguously relates a measured sensor response to a particular level of TAN and/or TBN. Next, the calibrated sensor is now installed in an asset and every time the sensor measures a response, the sensor (or computing assembly) consults with the calibration function on the correspondence of the measured sensor response to the exact value of TAN and/or TBN.

The multivariable sensor described herein can refer to a single sensor capable of producing multiple response signals that are not substantially correlated with each other and where these individual response signals from the multivariable sensor are further analyzed using multivariate analysis tools to construct response patterns of sensor exposure to different analytes at different concentrations. In one embodiment, multivariable or multivariate signal transduction is performed on the multiple response signals using multivariate analysis tools to construct a multivariable sensor response pattern. In certain embodiments, the multiple response signals comprise a change in a real and imaginary parts of impedance of a sensing region in an operational contact with oil. In certain embodiments, the multiple response signals comprise a change in a capacitance and a change in a resistance of a sensing material disposed on a multivariable sensor when exposed to an analyte. In other embodiments, the multiple response signals comprise a change in a capacitance, a change in a resistance, a change in an inductance, or any combination thereof.

In other embodiments, a single (or univariate) response signal can be used to monitor TBN or TAN. This response signal can originate from a sensor. This response signal can comprise a change in a capacitance, a change in a resistance, a change in an inductance, or any change correlated to TBN or TAN. A separate other sensor can be used in combination with a TBN or TAN univariate response sensor to correct for environmental effects not correlated with TBN or TAN. Nonlimiting examples of such environmental effects include ambient temperature, humidity, pressure, and other known effects.

Multivariate analysis includes a mathematical procedure that is used to analyze more than one variable from the sensor response and to provide the information about the type of at least one environmental parameter from the measured sensor parameters and/or to provide quantitative information about the level of at least one environmental parameter from the measured sensor parameters. Non-limiting examples of multivariate analysis tools include canonical correlation analysis, regression analysis, nonlinear regression analysis, principal components analysis, discriminate function analysis, multidimensional scaling, linear discriminate analysis, logistic regression, or neural network analysis.

Alternative or complementary to multivariate analysis, machine learning can be used. Machine learning includes mathematical procedures that automate creation of analytical models. These analytical models may predict future outcomes of TBN or TAN change or change of another parameter or other parameters of interest and change of the engine or another access using collected data. Machine learning is the approach to build and implement predictive algorithms to achieve these goals. Non-limiting examples of machine learning tools may include classification, regression, dimensionality reduction, preprocessing, model selection, clustering, decision tree learning, association rule learning, artificial neural networks, support vector machines, bayesian networks, genetic algorithms, and others.

Spectral parameters include measurable variables of the sensor response. The sensor response is the impedance spectrum of the LCR sensor. In another embodiment, the sensor response is the impedance spectrum of the non-resonant impedance sensor. In yet another embodiment, the sensor response is the optical spectrum in infrared, near-infrared, visible or ultraviolet range of spectrum. In addition to measuring the impedance spectrum in the form of Z-parameters, S-parameters, and other parameters, the impedance spectrum (for example, both real and imaginary parts) may be analyzed simultaneously using various parameters for analysis, such as, the frequency of the maximum of the real part of the impedance (Fp), the magnitude of the real part of the impedance (Zp), the resonant frequency of the imaginary part of the impedance (F1), the anti-resonant frequency of the imaginary part of the impedance (F2), signal magnitude (Z1) at the resonant frequency of the imaginary part of the impedance (F1), signal magnitude (Z2) at the anti-resonant frequency of the imaginary part of the impedance (F2), and zero-reactance frequency (Fz, frequency at which the imaginary portion of impedance is zero). Other spectral parameters may be simultaneously measured using the entire impedance spectra, for example, quality factor of resonance, phase angle, and magnitude of impedance. Spectral parameters calculated from the impedance spectra may also be called features or descriptors. The appropriate selection of features is performed from all potential features that can be calculated from spectra.

Sensing materials and sensing films include, but are not limited to, materials deposited onto a transducer's electronics circuit components, such as LCR circuit components to perform the function of predictably and reproducibly affecting the impedance sensor response upon interaction with the environment. In order to prevent the material in the sensor film from leaching into the liquid environment, the sensing materials are attached to the sensor surface using standard techniques, such as covalent bonding, electrostatic bonding, and other techniques.

One or more embodiments of the sensing systems described herein may measure the amount or concentration of basic (alkaline) and/or acidic components in engine oil and, based on the measurements, determine the corresponding TBN and/or TAN of the oil. The data acquisition circuitry may direct the sensor to measure the TBN and/or TAN repeatedly (e.g., on a continuous basis). The measured values of TBN and/or TAN may be periodically recorded by the computer 124 onto one or more memories. The frequency at which the TBN and/or TAN is determined and/or recorded may be based on the application (e.g., use) of the engine and/or the duty cycle of the engine. For example, the TBN and/or TAN of engines used for higher loads (e.g., engines operating to propel heavy vehicles) and/or used more often (e.g., over a wide range of engine operating conditions) may be measured or recorded more often than for engines used for lighter loads (e.g., lighter vehicles) and/or engines used less often (e.g., engines having fewer cycles).

The in-cylinder health of the engine can be monitored by measuring the amount of TBN and/or TAN in the oil that lubricates the engine. Normal lube oil consumption in the engine or a normal rate of oil consumption (e.g., within designated or predetermined limits) can indicate a healthy engine and healthy combustion within the engine. But, excess oil consumption or an excessive rate of oil consumption (e.g., outside of the designated or predetermined limits) can indicate an unhealthy engine and/or unhealthy combustion within the engine. Monitoring the TAN and/or TBN in the oil over time can allow for the engine to be proactively inspected or serviced before additional cylinder wear and/or seizing of cylinders within the engine. This can save the engine from major or significant repairs or catastrophic failure, and can reduce the life cycle cost of the engine. Monitoring the TBN and/or TAN changes can provide insight into the reliability and/or durability of the engine. The “unhealthy” state of a component such as an engine can be identified or determined responsive to an increase or decrease in the TBN or TAN, as appropriate and as described herein, relative to one or more previous measurements and/or by comparing the TBN or TAN measurement to one or more thresholds.

Although FIG. 3 illustrates a flowchart of one embodiment of a method 800 for monitoring lubricant (e.g., oil) health where at 864, measurement of electrical resonance parameters of the resonance spectra at several resonances of the sensor is performed, other sensors known in the art may be used at steps 864, 870, and 880, and other steps.

As described herein, the method 400 can involve tracking the rate of change in TBN and/or TAN in the oil of an engine. Changes in the TBN and/or TAN can be correlated to oil consumption based on sensor calibrations. If the TBN and/or TAN indicates that the oil consumption in the engine exceeds an allowable upper limit for a healthy engine, then the method 800 can involve servicing, inspecting, and/or repairing the engine. By examining the rates of change in the TBN and/or TAN (e.g., by using [d(TBN)/dt] and/or [d(TAN)/dt]), the method can be a self-learning method that accounts for engine-to-engine variability and that examines each engine as a separate or unique entity with its own inherent variations and/or tolerances. Optionally, tracking changes in TBN and/or TAN in one or more engines can be used to facilitate future engine designs.

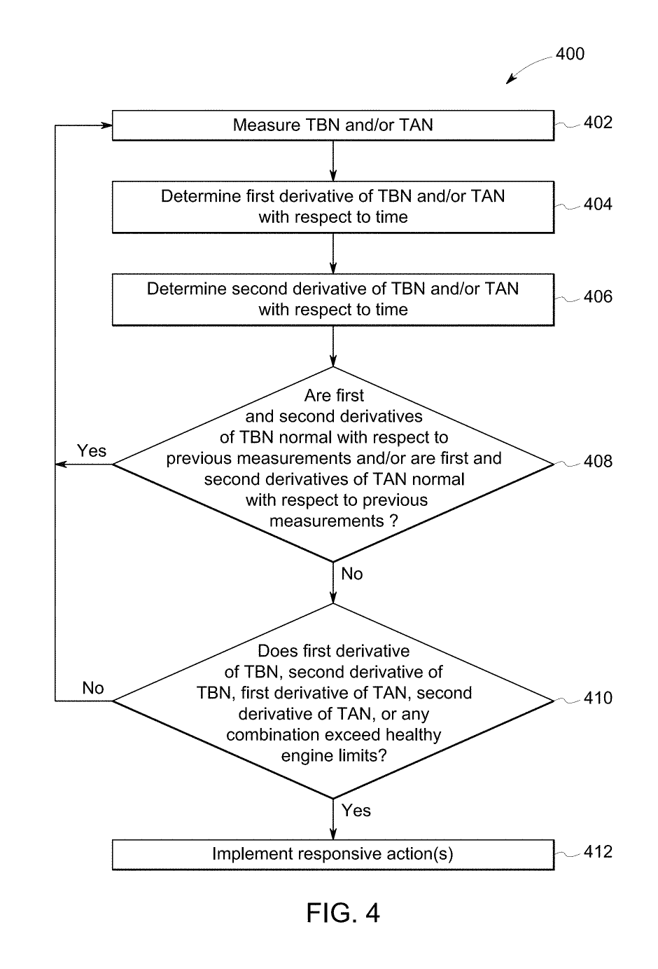

FIG. 4 illustrates a flowchart of one embodiment of a method 400 for monitoring lubricant (e.g., oil) health. The in-cylinder health of the engine can be monitored by measuring the amount of TBN and/or TAN in the oil that lubricates the engine. Normal lube oil consumption in the engine or a normal rate of oil consumption (e.g., within designated or predetermined limits) can indicate a healthy engine and healthy combustion within the engine. But, excess oil consumption or an excessive rate of oil consumption (e.g., outside of the designated or predetermined limits) can indicate an unhealthy engine and/or unhealthy combustion within the engine. Monitoring the TAN and/or TBN in the oil over time can allow for the engine to be proactively inspected or serviced before additional cylinder wear and/or seizing of cylinders within the engine. This can save the engine from major or significant repairs or catastrophic failure, and can reduce the life cycle cost of the engine. Monitoring the TBN and/or TAN changes can provide insight into the reliability and/or durability of the engine.

As described herein, the method 400 can involve tracking the rate of change in TBN and/or TAN in the oil of an engine. Changes in the TBN and/or TAN can be correlated to oil consumption based on sensor calibrations. If the TBN and/or TAN indicates that the oil consumption in the engine exceeds an allowable upper limit for a healthy engine, then the method 800 can involve servicing, inspecting, and/or repairing the engine. By examining the rates of change in the TBN and/or TAN (e.g., by using [d(TBN)/dt] and/or [d(TAN)/dt]), the method can be a self-learning method that accounts for engine-to-engine variability and that examines each engine as a separate or unique entity with its own inherent variations and/or tolerances. Optionally, tracking changes in TBN and/or TAN in one or more engines can be used to facilitate future engine designs.

At 402, TBN and/or TAN of oil in an engine is measured. One or more embodiments of the sensor described herein is at least partially immersed into the oil. Electrical resonance parameters of the resonance spectra at several resonances of the sensor is measured. For quantitation of contamination of engine oil by water, fuel leaks, and levels of TBN and/or TAN with a sensor, the sensor may be placed into operational contact with the fluid. In a specific embodiment, the resonant impedance spectra Ž(ƒ)=Zᵣₑ (ƒ)+jZᵢₘ(ƒ) of a sensor may be determined. For example, the parameters from the measured Ž(ƒ) spectra such as the frequency position Fₚ and magnitude Zₚ of Zᵣₑ(f) and the resonant F₁ and antiresonant F₂ frequencies, their magnitudes Z₁ and Z₂ of Zᵢₘ(f), and zero-reactance frequency F_{Z} of Zᵢₘ(ƒ), may be calculated. In another embodiment, the electrical resonance parameters may include capacitance parameters of the sensor in operational contact with the fluid, instead or in addition to impedance parameters. The measurements of TBN and/or TAN may be repeated multiple times in order to monitor for changes in TBN and/or TAN, as described herein.

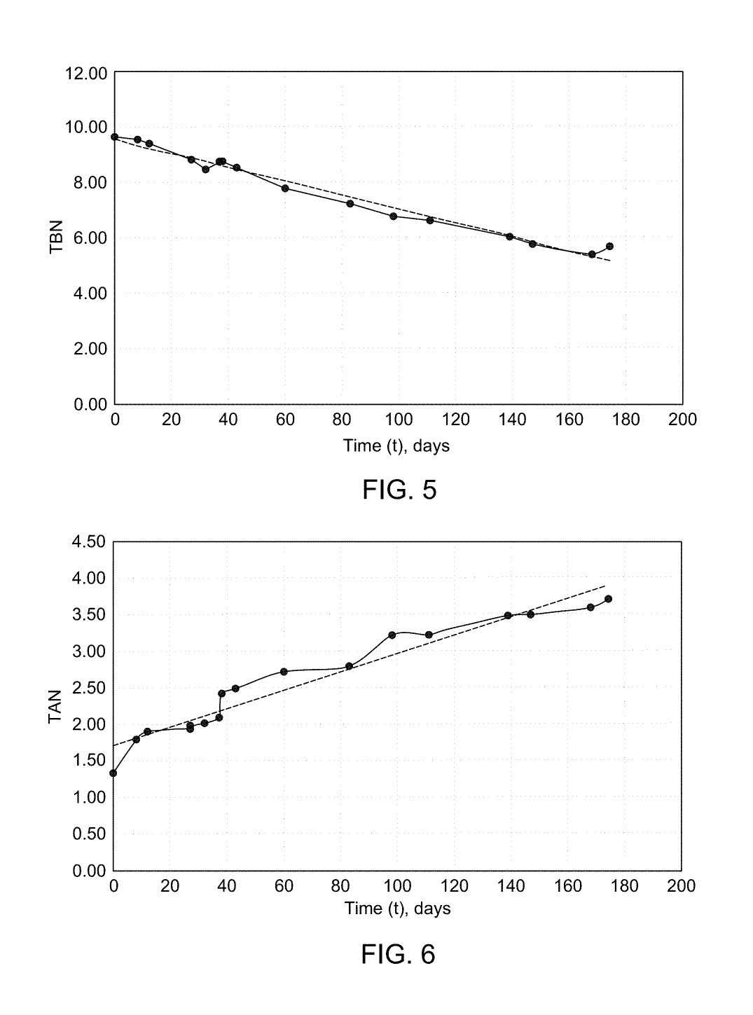

With continued reference to the flowchart of the method 400 shown in FIG. 4, FIGS. 5 and 6 illustrate measurements of TBN and TAN, respectively, according to one example. The TBN and TAN measurements are shown alongside horizontal axes indicative of time or number of measurements, and are shown alongside vertical axes indicative of the TBN and/or TAN measured in the oil. As shown in FIGS. 5 and 6, the TBN measurements may decrease over time while the TAN measurements may increase over time.

At 404 in the flowchart of the method 400, the first derivative of TBN and/or the first derivative of TAN with respect to time are determined. For example, the data acquisition circuitry 116 and/or the processor(s) of the computer 124 can calculate the change in TBN with respect to time (e.g., [d(TBN)/dt]) and/or the change in TAN with respect to time (e.g., [d(TAN)/dt]).

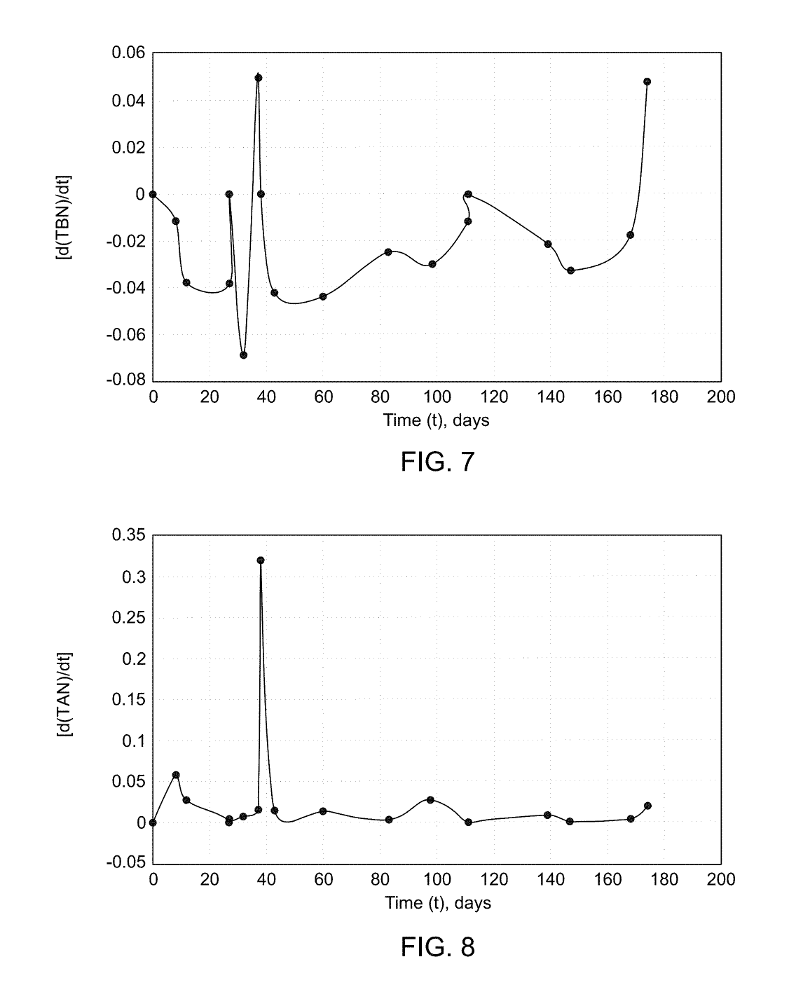

With continued reference to the flowchart of the method 400 shown in FIG. 4, FIGS. 7 and 8 illustrate first derivatives of TBN and TAN, respectively, with respect to time according to one example. The first derivatives of TBN and TAN are shown alongside horizontal axes indicative of time or number of measurements, and are shown alongside vertical axes indicative of the rates of change in the TBN and/or TAN. As shown in FIG. 7, the first derivative of TBN includes several peaks and valleys that indicate significant changes in the TBN measurements with respect to time. In contrast, the first derivative of TAN is relatively flat with a single large peak, which indicates that the TAN in the oil does not significantly change with respect to time except for the time at or near forty along the horizontal axis.

At 406 in the flowchart of the method 400, the second derivative of TBN and/or the second derivative of TAN with respect to time are determined. For example, the data acquisition circuitry 116 and/or the processor(s) of the computer 124 can calculate the second derivative of TBN with respect to time (e.g., [d²(TBN)/dt²]) and/or the change in TAN with respect to time (e.g., [d²(TAN)/dt²]).

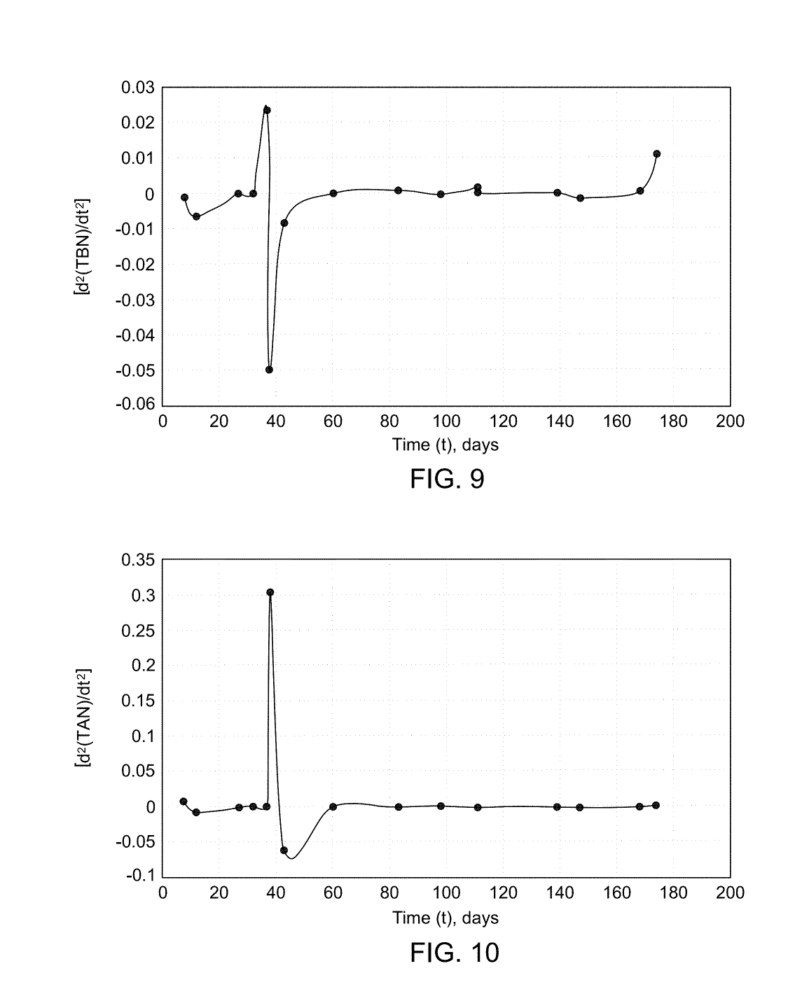

With continued reference to the flowchart of the method 400 shown in FIG. 4, FIGS. 9 and 10 illustrate second derivatives of TBN and TAN, respectively, with respect to time according to one example. The second derivatives of TBN and TAN are shown alongside horizontal axes indicative of time or number of measurements, and are shown alongside vertical axes indicative of the second derivatives of TBN and/or TAN.

At 408 in the flowchart of the method 400, a determination is made as to whether the first and/or second derivatives of TBN and/or the first and/or second derivatives of TAN are normal. This determination may be performed by the acquisition circuitry 116 and/or processor(s) of the computer 124 comparing the first and/or second derivatives of TBN and/or the first and/or second derivatives of TAN to one or more predetermined or previously designated limits.

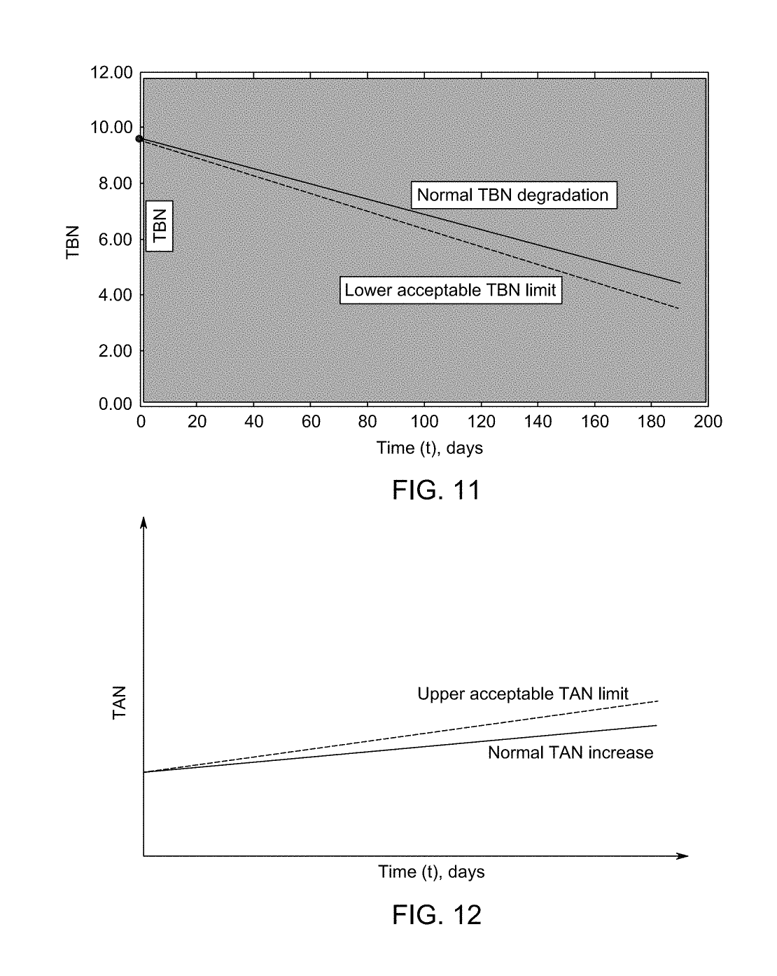

With continued reference to the flowchart of the method 400 shown in FIG. 4, FIG. 11 illustrates examples of predetermined or previously designated limits of TBN measurements. As shown in FIG. 11, TBN can normally degrade over time without indicating the need for repair, inspection, or maintenance of the engine. The limit on how the TBN can degrade over time is indicated by the “Normal TBN Degradation” line shown in FIG. 11, which is shown alongside a horizontal axis indicative of time or number of measurements and is shown alongside a vertical axis indicative of TBN measurements. Measurements of TBN that are above the normal TBN degradation limit or line indicate that the TBN in the oil is still within acceptable limits, and do not indicate a need to inspect, repair, or maintain the engine. A “Lower Acceptable TBN Limit” in FIG. 11 indicates a lower limit on the TBN measurements that indicates an unhealthy oil or engine. For example, TBN measurements that fall below this limit may indicate the need to repair, inspect, or maintain the engine, or to remove the engine from service.

FIG. 12 illustrates examples of predetermined or previously designated limits of TAN measurements. As shown in FIG. 12, TAN can normally increase over time without indicating the need for repair, inspection, or maintenance of the engine. The limit on how the TAN can increase over time is indicated by the “Normal TAN Increase” line shown in FIG. 12, which is shown alongside a horizontal axis indicative of time or number of measurements and is shown alongside a vertical axis indicative of TAN measurements. Measurements of TAN that are below the normal TAN increase limit or line indicate that the TAN in the oil is still within acceptable limits, and do not indicate a need to inspect, repair, or maintain the engine. An “Upper Acceptable TAN Limit” in FIG. 12 indicates an upper limit on the TAN measurements that indicates an unhealthy oil or engine. For example, TAN measurements that fall above this limit may indicate the need to repair, inspect, or maintain the engine, or to remove the engine from service.

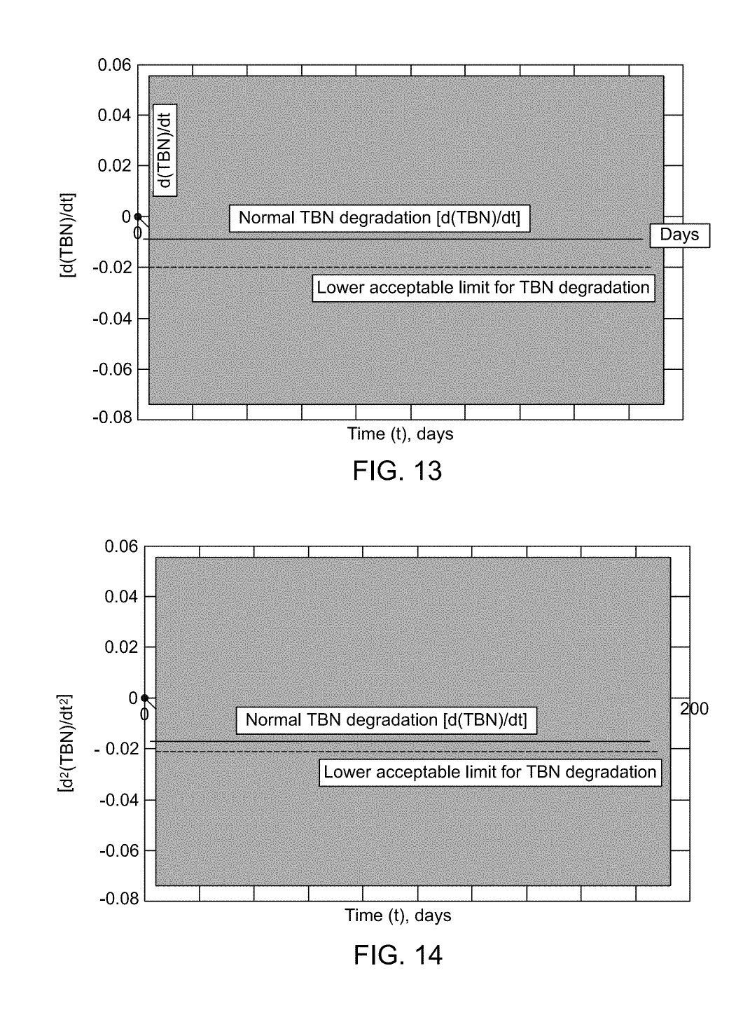

With respect to the determination that is made at 408 in the flowchart of the method 400 shown in FIG. 4, FIG. 13 illustrates examples of predetermined or previously designated limits of the first derivative of TBN. As shown in FIG. 13, a normal limit on the first derivative of TBN (“Normal TBN Degradation [d(TBN)/dt]” in FIG. 13) indicates a lower limit on the first derivative of TBN. First derivatives of TBN that are at or above this limit can be indicative of healthy oil or a healthy engine, and are not indicative of a need to repair, inspect, or maintain the engine, or to remove the engine from service, in one embodiment. Another, lower limit on the first derivative of TBN (“Lower Acceptable Limit for TBN Degradation” in FIG. 13) is described below.

FIG. 14 illustrates examples of predetermined or previously designated limits of the second derivative of TBN. As shown in FIG. 14, a normal limit on the second derivative of TBN (“Normal TBN Degradation [d(TBN)/dt]” in FIG. 14) indicates a lower limit on the second derivative of TBN. Second derivatives of TBN that are at or above this limit can be indicative of healthy oil or a healthy engine, and are not indicative of a need to repair, inspect, or maintain the engine, or to remove the engine from service, in one embodiment. Another, lower limit on the second derivative of TBN (“Lower Acceptable Limit for TBN Degradation” in FIG. 14) is described below.

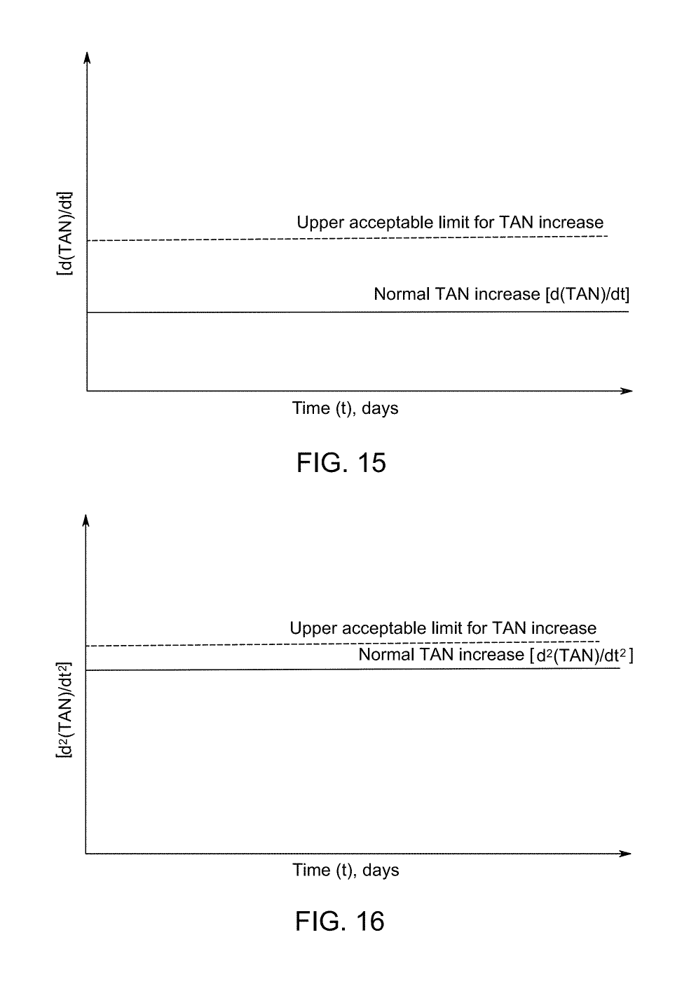

FIG. 15 illustrates examples of predetermined or previously designated limits of the first derivative of TAN. As shown in FIG. 15, a normal limit on the first derivative of TAN (“Normal TAN Increase [d(TAN)/dt]” in FIG. 15) indicates an upper limit on the first derivative of TAN. First derivatives of TAN that are at or below this limit can be indicative of healthy oil or a healthy engine, and are not indicative of a need to repair, inspect, or maintain the engine, or to remove the engine from service, in one embodiment. Another, greater limit on the first derivative of TAN (“Upper Acceptable Limit for TAN Increase” in FIG. 15) is described below.

FIG. 16 illustrates examples of predetermined or previously designated limits of the second derivative of TAN. As shown in FIG. 16, a normal limit on the second derivative of TAN (“Normal TAN Increase [d²(TBN)/dt²]” in FIG. 16) indicates an upper limit on the second derivative of TAN. Second derivatives of TAN that are at or below this limit can be indicative of healthy oil or a healthy engine, and are not indicative of a need to repair, inspect, or maintain the engine, or to remove the engine from service, in one embodiment. Another, greater limit on the second derivative of TAN (“Upper Acceptable Limit for TAN Increase” in FIG. 16) is described below.

With respect to the determination made at 408 in the method 400, if (a) the first derivative of TBN is greater than the associated normal limit (the upper of the two limits on the first derivative of TBN), (b) the first derivative of TAN is smaller than the associated normal limit (the lower of the two limits on the first derivative of TAN), (c) the second derivative of TBN is greater than the associated normal limit (the upper of the two limits on the second derivative of TBN), and (d) the second derivative of TAN is smaller than the associated normal limit (the lower of the two limits on the second derivative of TAN), then the first and second derivatives may indicate that the TBN and/or TAN of the oil are not outside of designated or predetermined (e.g., normal) limits. Optionally, the determination at 408 may determine whether a combination of two or more (but not all) of these first and/or second derivatives are above (e.g., for TBN) or below (e.g., for TAN) the associated normal limits described above. This can mean that the engine is not in need of repair, inspection, or maintenance, and that the engine can continue to operate. As a result, flow of the method 400 can return toward 402. Optionally, the method 400 can terminate.

But, if (a) the first derivative of TBN is smaller than the associated normal limit (the upper of the two limits on the first derivative of TBN), (b) the first derivative of TAN is greater than the associated normal limit (the lower of the two limits on the first derivative of TAN), (c) the second derivative of TBN is smaller than the associated normal limit (the upper of the two limits on the second derivative of TBN), and (d) the second derivative of TAN is greater than the associated normal limit (the lower of the two limits on the second derivative of TAN), then the first and second derivatives may indicate that the TBN and/or TAN of the oil are outside of designated or predetermined (e.g., normal) limits. Optionally, the determination at 408 may determine whether a combination of two or more of these first and/or second derivatives are below (e.g., for TBN) or above (e.g., for TAN) the associated normal limits described above. This can mean that the engine is in need of repair, inspection, or maintenance, and that the engine can no longer continue to safely operate. As a result, flow of the method 400 can proceed toward 410.

At 410, a determination is made as to whether the first and/or second derivatives of TBN and/or the first and/or second derivatives of TAN exceed healthy engine limits. This determination may be performed by the acquisition circuitry 116 and/or processor(s) of the computer 124 comparing the first and/or second derivatives of TBN and/or the first and/or second derivatives of TAN to one or more predetermined or previously designated limits. These limits (which differ from the normal limits used to make the determination at 408) can be referred to as acceptable limits.

For example, if (a) the first derivative of TBN is smaller than the associated acceptable limit (the lower of the two limits on the first derivative of TBN), (b) the first derivative of TAN is greater than the associated acceptable limit (the upper of the two limits on the first derivative of TAN), (c) the second derivative of TBN is smaller than the associated acceptable limit (the lower of the two limits on the second derivative of TBN), and (d) the second derivative of TAN is greater than the associated acceptable limit (the upper of the two limits on the second derivative of TAN), then the first and second derivatives may indicate that the TBN and/or TAN of the oil are outside of designated or predetermined (e.g., acceptable) limits. Optionally, the determination at 410 may determine whether a combination of two or more (but not all) of these first and/or second derivatives are below (e.g., for TBN) or above (e.g., for TAN) the associated acceptable limits. This can mean that the engine is in need of repair, inspection, or maintenance, and that the engine cannot continue to operate. As a result, flow of the method 400 can proceed toward 412.

But, if (a) the first derivative of TBN is not smaller than the associated acceptable limit, (b) the first derivative of TAN is not greater than the associated acceptable limit, (c) the second derivative of TBN is not smaller than the associated acceptable limit, and (d) the second derivative of TAN is not greater than the associated acceptable limit, then the first and second derivatives may indicate that the TBN and/or TAN of the oil are within the designated or predetermined (e.g., acceptable) limits. Optionally, the determination at 410 may determine whether a combination of two or more (but not all) of these first and/or second derivatives are not below (e.g., for TBN) or not above (e.g., for TAN) the associated acceptable limits. This can mean that the engine is not in need of repair, inspection, or maintenance, and that the engine can continue to operate. As a result, flow of the method 400 can return toward 402 or optionally terminate.

At 412, one or more responsive actions are implemented. These actions can include automatically stopping operation of the engine. For example, the computer 124 can generate a deactivation signal or instruct a vehicle controller to generate the deactivation signal that is communicated to fuel injectors of the engine and that directs the fuel injectors to stop providing fuel to the engine. As another example, the deactivation signal can otherwise control the engine, such as by decreasing (but not stopping) the supply of fuel to the engine from the fuel injectors. As another example, a control signal may be communicated to a facility to instruct the facility to inspect the engine and/or the oil, and potentially repair the engine or replace the oil.

Operation of the method 400 can be performed during operation of the engine to allow for monitoring and inspection of the health of the oil in the engine during operation of the engine. Instead of periodically collecting samples from the engine at times separated by many days (e.g., collecting samples every fifteen days), the sensor systems and methods described herein may collect measurements of characteristics of the oil on a much more frequent basis (e.g., once every second) or may continuously measure the characteristics of the oil. This can allow for very small changes in the health of the oil to be discovered immediately or very soon after the change occurs. This can allow for the engine to be repaired and/or the oil to be replaced or replenished before significant (e.g., catastrophic) damage to the engine occurs.

In one embodiment, a system includes a sensor configured to be in contact with oil within an engine of a vehicle system. The sensor includes a sensing region circuit that is configured to generate stimuli at different times during an operational life of the engine. The system also includes one or more processors configured to receive signals from the sensor. The signals are representative of responses of the oil to the stimuli. The one or more processors are configured to analyze the responses and determine a characteristic of the oil that represents one or more of a total base number (TBN) or a total acid number (TAN) of the oil. The one or more processors are configured to determine an unhealthy state of one or more of the engine or the oil based on the characteristic of the oil that is determined.

In one example, the sensor is one or more of an electrical sensor or an optical sensor.

In one example, the stimuli are one or more of electrical stimuli or optical stimuli.

In one example, the sensor is configured to continually generate the signals to the one or more processors such that the one or more processors are configured to continually monitor the one or more of the engine or the oil.

In one example, the one or more processors are configured to determine a rate of change in the TBN of the oil as the characteristic between a first time and a second time.

In one example, the one or more processors are configured to determine the unhealthy state of the one or more of the engine or the oil by comparing the rate of change in the TBN of the oil with a designated rate of change.

In one example, the one or more processors are configured to determine the unhealthy state of the one or more of the engine or the oil responsive to the rate of change in the TBN of the oil decreasing at a rate that is faster than the designated rate of change.

In one example, the one or more processors are configured to determine a rate of change in the TAN of the oil as the characteristic.

In one example, the one or more processors are configured to determine the unhealthy state of the one or more of the engine or the oil by comparing the rate of change in the TAN of the oil with a designated rate of change.

In one example, the one or more processors are configured to determine the unhealthy state of the one or more of the engine or the oil responsive to the rate of change in the TAN of the oil increasing at a rate that is faster than the designated rate of change.

In one example, the one or more processors are configured to determine a second derivative of the TBN of the oil as the characteristic.

In one example, the one or more processors are configured to determine the unhealthy state of the one or more of the engine or the oil by comparing the second derivative of the TBN of the oil with a predetermined second derivative.

In one example, the one or more processors are configured to determine the unhealthy state of the one or more of the engine or the oil responsive to the second derivative of the TBN of the oil changing at a rate that is faster than the designated second derivative.

In one example, the one or more processors are configured to determine a second derivative of the TAN of the oil as the characteristic.

In one example, the one or more processors are configured to determine the unhealthy state of the one or more of the engine or the oil by comparing the second derivative of the TAN of the oil with a designated second derivative.

In one example, the one or more processors are configured to determine the unhealthy state of the one or more of the engine or the oil responsive to the second derivative of the TAN of the oil changing at a rate that is faster than the designated second derivative.

In one example, the one or more processors are configured to determine the TBN of the oil as the characteristic.

In one example, the one or more processors are configured to determine the unhealthy state of the one or more of the engine or the oil by comparing the TBN of the oil with a designated TBN.

In one example, the one or more processors are configured to determine the unhealthy state of the one or more of the engine or the oil responsive to the TBN of the oil being smaller than the designated TBN.

In one example, the one or more processors are configured to determine the level of TAN of the oil as the characteristic.

In one example, the one or more processors are configured to determine the unhealthy state of the one or more of the engine or the oil by comparing the TAN of the oil with a designated TAN.

In one example, the one or more processors are configured to deactivate the engine responsive to determining the unhealthy state of the one or more of the engine or the oil based on the characteristic TBN and/or TAN of the oil that is determined.

In one example, the sensor includes a resonant circuit coupled with electrodes that are configured to generate an electric field between the electrodes as the stimuli with at least part of the lubricant disposed between the electrodes and within the electric field. At least one of the electrodes can include a dielectric coating. Alternatively, no coating is used. The resonant circuit can be configured to resonate at different frequencies responsive to generation of the electric field based on changes in the dielectric coating brought about by changes in one or more basic compounds or acidic compounds in the lubricant. Alternatively, the resonant circuit can resonate at different frequencies responsive to generation of the electric field based on changes in the concentration of basic compounds and/or acidic compounds in the lubricant in the electric field. The signals that are output from the sensor to the one or more processors can represent one or more of the frequencies at which the resonant circuit resonates. The one or more processors can be configured to compare the one or more frequencies at which the resonant circuit resonates with one or more designated frequencies associated with different TBN or TAN of the lubricant to determine the one or more of the TBN or the TAN of the lubricant.

In one embodiment, a method includes placing a sensor in contact with oil within an engine of a vehicle system, generating stimuli at a sensing region circuit of the sensor during an operational life of the engine, receiving signals from the sensor, the signals representative of responses of the oil to the stimuli, analyzing the responses to determine a characteristic of the oil that represents one or more of a total base number (TBN) or a total acid number (TAN) of the oil, and determining an unhealthy state of one or more of the engine or the oil based on the characteristic TBN and/or TAN of the oil that is determined.

In one example, the sensor is one or more of an electrical sensor or an optical sensor.

In one example, the signals are one or more of electrical signals or optical signals.

In one example, the stimuli are one or more of electrical stimuli or optical stimuli.

In one example, the responses are one or more of electrical responses or optical responses.

In one example, the signals are continually received from the sensor to continually monitor the one or more of the engine or the oil.

In one example, the characteristic that is determined is a rate of change in the TBN of the oil.

In one example, the unhealthy state of the one or more of the engine or the oil is determined by comparing the rate of change in the TBN of the oil with a designated rate of change.

In one example, the unhealthy state of the one or more of the engine or the oil is determined responsive to the rate of change in the TBN of the oil decreasing at a rate that is faster than the designated rate of change.

In one example, the characteristic that is determined is a rate of change in the TAN of the oil.

In one example, the unhealthy state of the one or more of the engine or the oil is determined by comparing the rate of change in the TAN of the oil with a designated rate of change.

In one example, the unhealthy state of the one or more of the engine or the oil is determined responsive to the rate of change in the TAN of the oil changing at a rate that is faster than the designated rate of change.

In one example, the characteristic is a second derivative of the TBN of the oil.

In one example, the unhealthy state of the one or more of the engine or the oil is determined by comparing the second derivative of the TBN of the oil with a designated second derivative.

In one example, the unhealthy state of the one or more of the engine or the oil is determined responsive to the second derivative of the TBN of the oil changing at a rate that is faster than the designated second derivative.

In one example, the characteristic is a second derivative of the TAN of the oil.

In one example, the unhealthy state of the one or more of the engine or the oil is determined by comparing the second derivative of the TAN of the oil with a designated second derivative.

In one example, the unhealthy state of the one or more of the engine or the oil is determined responsive to the second derivative of the TAN of the oil changing at a rate that is faster than the designated second derivative.

In one example, the characteristic is the TBN of the oil.

In one example, the unhealthy state of the one or more of the engine or the oil is determined by comparing the TBN of the oil with a designated TBN.

In one example, the unhealthy state of the one or more of the engine or the oil is determined responsive to the TBN of the oil being smaller than the designated TBN.

In one example, the characteristic is the TAN of the oil.

In one example, the unhealthy state of the one or more of the engine or the oil is determined by comparing the TAN of the oil with a designated TAN.

In one example, the method also includes deactivating the engine responsive to determining the unhealthy state of the one or more of the engine or the oil based on the characteristic TBN and/or TAN of the oil that is determined.

In one embodiment, a system includes a sensor configured to be in contact with lubricating oil within a rotating equipment of a system. The sensor is configured to generate detectable stimuli at different times during an operational life of the rotating equipment. The system also includes one or more processors configured to receive signals from the sensor. The signals are representative of responses of the oil to the stimuli. The one or more processors are configured to analyze the responses and determine a characteristic of the oil that represents one or more of a total base number (TBN) or a total acid number (TAN) of the oil. The one or more processors are configured to determine an unhealthy state of one or more of the engine or the oil based on the characteristic of the oil that is determined.

In one example, the sensor is an electrical, resonant, non-resonant, optical, and/or mechanical sensor.

In one example, the stimuli are electrical or optical stimuli.

In one embodiment, a method includes placing a sensor in contact with oil within an engine of a vehicle system, generating stimuli at a sensing region circuit of the sensor during an operational life of the engine, receiving signals from the sensor, the signals representative of responses of the oil to the stimuli, analyzing the responses to determine a characteristic of the oil that represents one or more of a total base number (TBN) or a total acid number (TAN) of the oil, and determining health state of one or more of the engine or the oil based on the characteristic of the oil that is determined.

In one example, the sensor is an electrical, resonant, non-resonant, optical, and/or mechanical sensor.

In one example, the stimuli are electrical or optical stimuli.

In one embodiment, a method includes generating stimuli at a sensing region circuit of a sensor that is in contact with oil associated with an engine system, receiving signals from the sensor, the signals representative of responses of the oil to the stimuli, analyzing the responses to determine a characteristic of the oil that represents one or more of a total base number (TBN) or a total acid number (TAN) of the oil, performing a comparison of the characteristic of the oil that is determined to one or more designated corresponding characteristic thresholds that are indicative of oil conditions, and controlling the engine system based at least in part on the comparison. The engine system can be controlled in a variety of different manners based on results of the comparison. For example, for some TBN measurements, TAN measurements, changes in TBN or TAN measurements, etc., the engine system can be controlled by restricting a power output of the engine system. For other TBN measurements, TAN measurements, changes in TBN or TAN measurements, etc., the engine system can be controlled by automatically shutting off or deactivating the engine system. For other TBN measurements, TAN measurements, changes in TBN or TAN measurements, etc., the engine system can be controlled by automatically restricting how rapidly changes in the power output of the engine system can occur.

As used herein, an element or step recited in the singular and proceeded with the word “a” or “an” should be understood as not excluding plural of said elements or steps, unless such exclusion is explicitly stated. Furthermore, references to “one embodiment” of the presently described inventive subject matter are not intended to be interpreted as excluding the existence of additional embodiments that also incorporate the recited features. Moreover, unless explicitly stated to the contrary, embodiments “comprising,” “including,” or “having” (or like terms) an element, which has a particular property or a plurality of elements with a particular property, may include additional such elements that do not have the particular property.

As used herein, terms such as “system” or “controller” may include hardware and/or software that operate(s) to perform one or more functions. For example, a system or controller may include a computer processor or other logic-based device that performs operations based on instructions stored on a tangible and non-transitory computer readable storage medium, such as a computer memory. Alternatively, a system or controller may include a hard-wired device that performs operations based on hard-wired logic of the device. The systems and controllers shown in the figures may represent the hardware that operates based on software or hardwired instructions, the software that directs hardware to perform the operations, or a combination thereof.

As used herein, terms such as “operably connected,” “operatively connected,” “operably coupled,” “operatively coupled” and the like indicate that two or more components are connected in a manner that enables or allows at least one of the components to carry out a designated function. For example, when two or more components are operably connected, one or more connections (electrical and/or wireless connections) may exist that allow the components to communicate with each other, that allow one component to control another component, that allow each component to control the other component, and/or that enable at least one of the components to operate in a designated manner.

It is to be understood that the subject matter described herein is not limited in its application to the details of construction and the arrangement of elements set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the presently described subject matter without departing from its scope. While the dimensions, types of materials and coatings described herein are intended to define the parameters of the disclosed subject matter, they are by no means limiting and are exemplary embodiments. Many other embodiments will be apparent to one of ordinary skill in the art upon reviewing the above description. The scope of the inventive subject matter should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

This written description uses examples to disclose several embodiments of the inventive subject matter, and also to enable one of ordinary skill in the art to practice the embodiments of inventive subject matter, including making and using any devices or systems and performing any incorporated methods. The patentable scope of the inventive subject matter is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

What is claimed is:

1. A system comprising:
a sensor configured to be in contact with lubricant within an engine, the sensor including a sensing region circuit that is configured to generate stimuli at different times during an operational life of the engine; and
one or more processors configured to receive signals from the sensor, the signals representative of responses of the lubricant to the stimuli, the one or more processors configured to analyze the responses and determine a characteristic of the lubricant that represents one or more of a total base number (TBN) or a total acid number (TAN) of the lubricant,
wherein the one or more processors are configured to determine an unhealthy state of one or more of the engine or the lubricant based on the characteristic of the lubricant that is determined.

2. The system of claim 1, wherein the sensor comprises one or more of an electrical sensor or an optical sensor and the stimuli include one or more of electrical stimuli or optical stimuli.

3. The system of claim 1, wherein the one or more processors are configured to determine one or more of a rate of change in the TBN or a rate of change in the TAN of the lubricant as the characteristic between a first time and a second time.

4. The system of claim 3, wherein the one or more processors are configured to determine the unhealthy state of the one or more of the engine or the lubricant responsive to one or more of the rate of change in the TBN of the lubricant decreasing at a rate that is faster than a designated rate of change or the rate of change in the TAN increasing at a rate that is faster than the designated rate of change.

5. The system of claim 1, wherein the one or more processors are configured to determine one or more of a second derivative of the TBN of the lubricant or a second derivative of the TAN of the lubricant as the characteristic.

6. The system of claim 5, wherein the one or more processors are configured to determine the unhealthy state of the one or more of the engine or the lubricant responsive to one or more of the second derivative of the TBN of the lubricant changing at a rate that is faster than a designated second derivative or the second derivative of the TAN of the lubricant changing at a rate that is faster than the designated second derivative.

7. The system of claim 1, wherein the one or more processors are configured to determine one or more of the TBN or the TAN of the lubricant as the characteristic.

8. The system of claim 7, wherein the one or more processors are configured to determine the unhealthy state of the one or more of the engine or the lubricant responsive to one or more of the TBN of the lubricant being smaller than a designated TBN or the TAN of the lubricant being larger than a designated TAN.

9. The system of claim 1, wherein the one or more processors are configured to deactivate or otherwise control the engine or a vehicle in which the engine is disposed responsive to determining the unhealthy state of the one or more of the engine or the lubricant based on one or more of the TBN or the TAN of the lubricant that is determined.

10. The system of claim 1, wherein the sensor includes a resonant circuit coupled with electrodes that are configured to generate an electric field between the electrodes as the stimuli with at least part of the lubricant disposed between the electrodes and within the electric field,
wherein the resonant circuit is configured to resonate at different frequencies responsive to generation of the electric field based on a concentration of one or more basic compounds or acidic compounds in the lubricant between the electrodes,
wherein the signals that are output from the sensor to the one or more processors represents one or more of the frequencies at which the resonant circuit resonates,
wherein the one or more processors are configured to compare the one or more frequencies at which the resonant circuit resonates with one or more designated frequencies associated with different TBN or TAN of the lubricant to determine the one or more of the TBN or the TAN of the lubricant.

11. A method comprising:
generating stimuli at a sensing region circuit of a sensor during an operational life of an engine, wherein the sensor is in contact with lubricant associated with the engine;
receiving signals from the sensor, the signals representative of responses of the lubricant to the stimuli;
analyzing the responses to determine a characteristic of the lubricant that represents one or more of a total base number (TBN) or a total acid number (TAN) of the lubricant; and
determining an unhealthy state of one or more of the engine or the lubricant based on the characteristic TBN and/or TAN of the lubricant that is determined.

12. The method of claim 11, wherein the characteristic that is determined is one or more of a rate of change in the TBN of the lubricant or a rate of change in the TAN of the lubricant.

13. The method of claim 12, wherein the unhealthy state of the one or more of the engine or the lubricant is determined responsive to one or more of the rate of change in the TBN of the lubricant decreasing at a rate that is faster than a designated rate of change or the rate of change in the TAN of the lubricant increasing at a rate that is faster than the designated rate of change.

14. The method of claim 11, wherein the characteristic is one or more of a second derivative of the TBN of the lubricant or a second derivative of the TAN of the lubricant.

15. The method of claim 11, wherein the unhealthy state of the one or more of the engine or the lubricant is determined responsive to one or more of the second derivative of the TBN of the lubricant changing at a rate that is faster than a designated second derivative or the second derivative of the TAN of the lubricant changing at a rate that is faster than the designated second derivative.

16. The method of claim 11, wherein the characteristic is one or more of the TBN of the lubricant or the TAN of the lubricant.

17. The method of claim 16, wherein the unhealthy state of the one or more of the engine or the lubricant is determined by comparing one or more of the TBN of the lubricant with a designated TBN or the TAN of the lubricant with a designated TAN.

18. The method of claim 11, further comprising deactivating or otherwise controlling the engine or a vehicle in which the engine is disposed responsive to determining the unhealthy state of the one or more of the engine or the lubricant based on one or more of the TBN of the lubricant or the TAN of the lubricant that is determined.

19. A system comprising:
a sensor configured to be in contact with a lubricant within a rotating equipment of a system, wherein the sensor is configured to generate detectable stimuli at different times during an operational life of the rotating equipment; and
one or more processors configured to receive signals from the sensor, the signals representative of responses of the lubricant to the stimuli, the one or more processors configured to analyze the responses and determine a characteristic of the lubricant that represents one or more of a total base number (TBN) or a total acid number (TAN) of the lubricant,
wherein the one or more processors are configured to determine an unhealthy state of one or more of the engine or the lubricant based on the characteristic of the lubricant that is determined.

20. The system of claim 19, wherein the sensor comprises one or more of an electrical, resonant, non-resonant, optical, or mechanical sensor.
